(19) United States
(12) Patent Application Publication
(10) Pub. No.: US 2008/0095303 A1
(43) Pub. Date: Apr. 24, 2008

(54) Apparatus For The Evaluation Of Rotational X-Ray Projections

(75) Inventors: Michael Grass, Buchholz in der Nordheide (DE); Volker Rasche, Wellesley, MA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/573,577

(22) PCT Filed: Aug. 17, 2005
(86) PCT No.: PCT/IB05/52714

§ 371 Date: Feb. 12, 2007

(30) Foreign Application Priority Data
Aug. 18, 2004 (EP) 04300546.1

(51) Int. Cl. A61B 6/03

(52) U.S. Cl. 378/5

(57) ABSTRACT

The invention relates to a method and an examination apparatus for the evaluation of X-ray projections (31-33, 41-43) that were generated with a rotational X-ray device (10) from different directions and with an energy level varying periodically from projection to projection. Said variation may for example be achieved by a continuously modulated tube voltage (V). Two different 3D-images (35, 45) may be reconstructed from the X-ray projections (31-33, 41-43) which belong to the different energy levels, and said 3D-images may then be compared voxel by voxel in order to segment structures (50) of interest due to contrast differences.

### FIELD OF THE INVENTION

The invention relates to an examination apparatus with a rotational X-ray device for the generation of X-ray projections of a body volume from a sequence of different directions and to a corresponding method for the generation of three-dimensional images of a body volume.

### BACKGROUND OF THE INVENTION

From the U.S. Pat. No. 4,361,901 an X-ray tube is known with a special design for fast switching of the tube voltage between two or more different levels, wherein said switching allows the generation of X-ray projections with different energy levels of the X-ray quanta. Due to the energy dependent absorption behavior of different materials in a body volume, different structures in the body are represented differently in said X-ray projections. This effect can be used to generate difference images in which certain structures, particularly blood vessels filled with a contrast agent, are represented with a high contrast.

### SUMMARY OF THE INVENTION

Based on this situation it was an object of the present invention to provide means for an improved visualization of and discrimination between different structures of a body volume.

This object is achieved by an examination apparatus according to claim 1 and by a method according to claim 9. Preferred embodiments are disclosed in the dependent claims.

The examination apparatus according to the present invention comprises the following components:
A rotational X-ray device that is adapted to acquire X-ray projections of a body volume from a (preferably ordered) sequence of different directions, for example in one run of a continuous movement from a (semi-) circle around the object, wherein said projections are generated with a periodically varying energy level of the X-ray quanta. A “varying energy level” means, more strictly speaking, that the spectra of the X-rays used to generate the X-ray projections are different, wherein each spectrum can be associated with a characteristic energy level (for example the average or the maximal energy of the spectrum). In the case of monochromatic X-rays, the spectra are for example degenerated to lines with just one energy. The energy level may particularly switch back and forth between two values from projection to projection, i.e. having a first value E₁ for projections with an odd serial number and a different second value E₂ for projections with an even serial number.

An image processing device, for example a computer, that is adapted to reconstruct at least two three-dimensional (3D) images of the body volume from X-ray projections that were generated by the aforementioned X-ray device from a sequence of different directions, wherein the projections used for the reconstruction of each 3D-image correspond to different energy levels. Moreover, the image processing device is adapted to segment structures of interest, for example blood vessels, based on a comparison of corresponding voxels in the aforementioned 3D-images. As usual, “segmentation” denotes in this context the process of classifying or associating picture elements (pixels, voxels) of an image to different objects or classes.

The described examination apparatus allows to determine and visualize three-dimensionally structures in a body volume that have only a very low contrast in X-ray projections or in a three-dimensional reconstructed volume. This result is achieved by the application of X-radiation with different energy levels in a rotational X-ray apparatus, thus generating series of projections which are suited for the reconstruction of energy dependent 3D-images. Because said 3D-images are obtained from interleaved X-ray projections, the geometries of the represented body volumes are highly identical (and for example do not differ due to body motions of the patient). The high geometric agreement between the generated 3D-images makes it possible to compare said images voxel by voxel and thus to segment structures of interest based on their different contrast in the different 3D-images. It should also be noted that the step of segmentation comprises more than the usual generation of a subtraction image, because segmentation implies the association of voxels with different objects. The result of the segmentation procedure may thus be an isolated or binary structure, for example a vessel tree in three dimensions.

The X-ray device of the examination apparatus may particularly comprise an X-ray tube and an X-ray detector that are coupled to a common carrier, for example a C-arm, which can be rotated about an axis or a point. X-ray devices of this kind are often already present in conventional X-ray examination laboratories.

The generation of X-rays with different energy levels may be achieved in different ways, for example by changing filters in the path of a constant radiation. Preferably, the varying energy levels are generated by an X-ray tube of the X-ray device that is adapted to generate X-rays with a periodically varying tube voltage. Higher tube voltages then generate X-ray quanta of higher energy, wherein the desired energy levels and the temporal course of the energy variation can be readily controlled by the tube voltage.

According to a first special realization of the aforementioned embodiment, the tube voltage switches sequentially between two or more discrete voltage levels, i.e. the voltage changes in steps.

According to a second realization, the tube voltage is modulated continuously, for example according to the course of a sinusoidal function (with an offset). Such a continuous modulation has the advantage that its generation may be easier.

In a further development of the examination apparatus, the image processing device may be adapted to reconstruct a three-dimensional image based on all available X-ray projections (i.e. irrespective of the energy level with which they were generated). Such a use of all available data allows the reconstruction of three-dimensional images with higher spatial resolution.

In the aforementioned apparatus, the high resolution three-dimensional image may optionally be masked with at least one of the low resolution three-dimensional images or with a new data set derived from said two low resolution images (for example on a per-voxel basis), said masking being followed by a subsequent segmentation of the high resolution image. The new data set may in the most simple case be the voxel-wise difference between the two low resolution images. Furthermore, the new data set may be segmented and adjusted to the higher resolution of the high resolution 3D image and then be used to segment this 3D image. Alternatively, the new data set may be adjusted to the higher resolution first, and the segmentation may be based on information taken from the data sets with higher and lower resolution. The advantage of the aforementioned approaches is that a high spatial resolution may be combined with an improved segmentation.

The apparatus furthermore optionally comprises a display unit for the display of reconstructed 3D-images and/or of processing results thereof, for example of the three-dimensional segmented structures.

The invention further relates to a method for the generation of three-dimensional images of a body volume, the method comprising the following steps:
Generating X-ray projections from a sequence of different directions, wherein said projections are generated (preferably interleaved) with a periodically varying energy level of the X-ray quanta, resulting in at least two projection data sets corresponding to different X-ray energies;
Reconstructing at least two three-dimensional images of the body volume from X-ray projections of said data sets that correspond to different energy levels.
Segmenting structures of interest based on a comparison of corresponding voxels in the 3D-images.

The method comprises in general form the steps that can be executed with an examination apparatus of the kind described above. Therefore, reference is made to the preceding description for more information on the details, advantages and improvements of that method.

According to a preferred embodiment of the method, the X-radiation is generated by an X-ray tube with a continuously modulated tube voltage.

The values of the different energy levels that are used for the generation of X-ray projections are preferably adjusted to the structure or the material that are of particular interest and that shall be segmented. It is especially possible to choose at least one energy level of the X-ray quanta above and at least one other energy level below an absorption edge (K-edge) of a contrast agent that is present in the imaged body volume. In this case the X-radiation with the higher energy level will be absorbed by the contrast agent while the radiation with the lower energy level will not, thus yielding a high contrast between the corresponding projections.

These and other aspects of the invention will be apparent from and elucidated with reference to the embodiment(s) described hereinafter.

### BRIEF DESCRIPTION OF THE DRAWINGS

In the following the invention is described by way of example with the help of the accompanying drawings in which:

FIG. 1 schematically depicts an examination apparatus according to the present invention for the segmentation of blood vessels in a 3D X-ray image of a body volume;

FIG. 2 represents a flow chart of the method according to the present invention.

### DESCRIPTION OF PREFERRED EMBODIMENTS

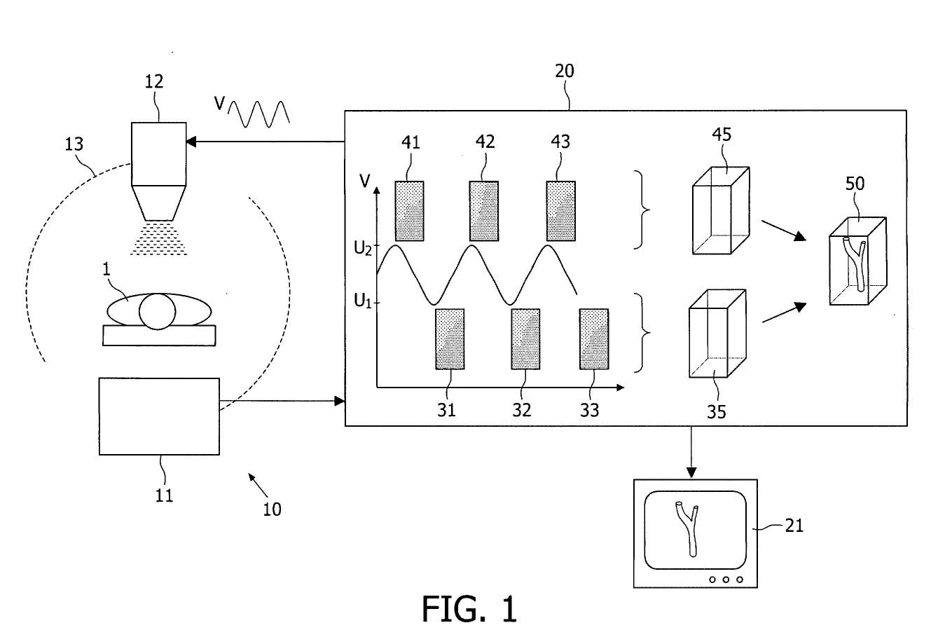

In the left part of FIG. 1 a rotational X-ray device 10 comprising an X-ray tube 12 and an X-ray detector 11 is schematically shown. The tube 12 and the detector 11 are mechanically coupled and can be rotated along an arc 13 around a patient 1 lying on a table in the centre of the device 10. The usual application of such a rotational X-ray device 10 comprises the generation of X-ray projections with X-radiation of a certain spectrum or energy level from different directions along the arc 13, wherein said projections are passed on to an image processing device 20 that is able to reconstruct a 3D-image thereof. Before the generation of the X-ray projections a contrast agent may be injected into the vessel system of the patient 1 in order to increase the visibility of the vessels on the projections (called “three-dimensional rotational angiography” or 3D-RA).

One of the basic problems in 3D-RA imaging is the volumetric visualization and segmentation of the contrast agent enhanced vascular systems. In the area of neuroradiology, this problem is extremely hard to solve, since the values and spatial positions for voxels containing bony structures and contrast agent filled vessels can be quite similar. Pure segmentation methods fail and combined segmentation and region growing approaches cannot handle this either.

To solve the aforementioned problems it is suggested here to acquire the X-ray projections of a 3D-RA run by switching the spectrum of the X-rays between two or more different energies from view to view. Such a switching of X-ray energies may particularly be achieved by a continuously modulated tube voltage V, wherein an image is for example generated each time the voltage passes a local maximum U₂ or minimum U₁ or any voltage chosen in between.

The image processing device 20 may be a computer comprising usual components like central processing unit, volatile and nonvolatile memory, I/O-interfaces and the like together with appropriate software. In FIG. 1, not these hardware components but a schematic representation of the processing steps executed by the device 20 is shown. As described above, the image processing device 20 is provided with (at least) one set of projections 41, 42, 43, . . . that were generated with X-radiation of a higher energy (high tube voltage U₂), and a second set of X-ray projections 31, 32, 33, . . . that were generated with X-radiation of the lower energy (lower tube voltage U₁). Both data sets can then be used for the reconstruction of a complete volume 35 and 45 each. As the X-ray projections on which said 3D-images are based are interleaved, the 3D-images 35, 45 represent the same geometry. Due to the different energy levels used for the generation of the 3D-images 35, 45, the contrast with which different structures are represented in said 3D-images is however different according to the energy dependent X-ray absorption characteristics. In a further step, these different values of each voxel in the 3D-images 35, 45 are then used to characterize different structures like bone or vessel. Thus a structure of interest, e.g. a vessel tree, can be segmented in three dimensions to produce the segmentation image 50, wherein the result of said segmentation may optionally be displayed on a monitor 21 coupled to the image processing device 20.

The whole set of X-ray projections 31-33, 41-43 may optionally be used to reconstruct a combined 3D-image (not shown) with improved radial sampling and high spatial resolution. The low-resolution data sets 31-33 and 41-43, respectively, (or any other data set derived thereof, e.g. the 3D-images 35, 45) may then further be used to mask said high resolution 3D-image for a subsequent segmentation.

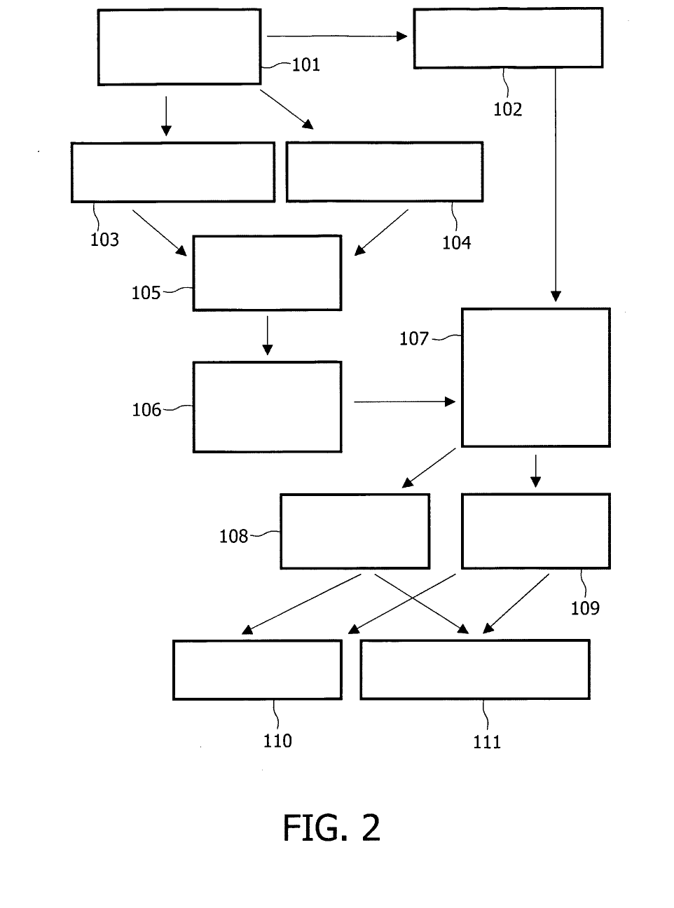

FIG. 2 additionally shows a flow chart of an examination procedure that can be executed with the examination apparatus described above, wherein the blocks of the chart represent the following steps:
101 Rotational projection acquisition with two energies U₁<U₂ switching from view to view
102 3D cone beam reconstruction of the whole volume Vₐₗₗ from all projections
103 3D cone beam reconstruction of the volume 35 from projections acquired with U₁
104 3D cone beam reconstruction of the volume 45 from projections acquired with U₂
105 Comparison of energy dependent contrast values per voxel from volumes 35, 45
106 Characterization of voxel as contrast agent filled vessel and bone due to contrast change
107 Segmentation of contrast agent filled vessels or the bony structures due to characterization and additional parameters (threshold/shape/region growing)
108 Segmented high resolution volume containing bony structure only: V^{B}ₐₗₗ
109 Segmented high resolution volume containing vascular structure only V^{V}ₐₗₗ
110 Visualization of volumes or V^{B}ₐₗₗ and V^{V}ₐₗₗ separately
111 Combined visualization of volumes or V^{B}ₐₗₗ and V^{V}ₐₗₗ with different color maps/weighting.

Finally it is pointed out that in the present application the term “comprising” does not exclude other elements or steps, that “a” or “an” does not exclude a plurality, and that a single processor or other unit may fulfill the functions of several means. Moreover, reference signs in the claims shall not be construed as limiting their scope.

1. Examination apparatus, comprising
a rotational X-ray device (10) that is adapted to generate X-ray projections (31-33, 41-43) of a body volume (1) from a sequence of different directions and with a periodically varying energy level of the X-ray quanta;
an image processing device (20) that is adapted to reconstruct at least two 3D-images (35, 45) of a body volume (1) from X-ray projections (31-33, 41-43) that were generated by said X-ray device (10) from a sequence of different directions and that correspond to different energy levels, the image processing device (20) further being adapted to segment structures (50) of interest based on a comparison of corresponding voxels in the 3D-images (35, 45).

2. The examination apparatus according to claim 1, characterized in that the X-ray device (10) comprises an X-ray tube (12) and an X-ray detector (11) that are coupled to a common carrier that can be rotated about and axis or about a point.

3. The examination apparatus according to claim 1, characterized in that the X-ray device (10) comprises an X-ray tube (12) that is adapted to generate X-rays with a periodically varying tube voltage (V).

4. The examination apparatus according to claim 3, characterized in that the tube voltage switches sequentially between two or more levels.

5. The examination apparatus according to claim 3, characterized in that the tube voltage (V) is modulated continuously.

6. The examination apparatus according to claim 1, characterized in that the image processing device (20) is adapted to reconstruct a further 3D-image based on all available X-ray projections (31-33, 41-43).

7. The examination apparatus according to claim 6, characterized in that the image processing device (20) is adapted to segment said further 3D-image based on a masking with one of the at least two 3D-images (35, 45) or a data set derived thereof.

8. The examination apparatus according to claim 1, characterized in that it comprises a display unit (21) for the display of reconstructed 3D-images (35, 45) and/or of processing results obtained thereof.

9. A method for the generation of 3D-images (35, 45) of a body volume (1), comprising the steps of:
generating X-ray projections (31-33, 41-43) from a sequence of different directions and with a periodically varying energy level of the X-ray quanta;
reconstructing at least two 3D-images (35, 45) of a body volume (1) from said X-ray projections (31-33, 41-43) that correspond to different energy levels;
segmenting structures (50) of interest based on a comparison of corresponding voxels in the 3D-images (35, 45).

10. The method according to claim 9, characterized in that X-radiation is generated by an X-ray tube (12) with a continuously modulated tube voltage (V).

11. The method according to claim 9, characterized in that at least one energy level of the X-ray quanta is above and one energy level below an absorption edge of a contrast agent present in the body volume.
